(19) United States
(12) Patent Application Publication
(10) Pub. No.: US 2012/0107257 A1
(43) Pub. Date: May 3, 2012

(54) PIGMENTARY AQUEOUS DISPERSION AND COSMETIC USES THEREOF

(75) Inventors: Alexandre CAPPON, Clichy (FR); Patricia Vesque, Franconville (FR); Gaelle Frere, Compiegne (FR)

(73) Assignee: SENSIENT COSMETIC TECHNOLOGIES, Saint-Quen l'Aumone (FR)

(21) Appl. No.: 13/282,232

(22) Filed: Oct. 26, 2011

(30) Foreign Application Priority Data
Oct. 27, 2010 (FR) 10 58843

(51) Int. Cl. A61K 8/81; C08K 3/22; C08K 5/3417; A61Q 1/10; C08K 5/053; A61K 8/04; A61Q 5/06; C08L 39/06; C08K 3/04

(52) U.S. Cl. 424/63; 524/548; 524/413; 524/432; 524/431; 524/407; 524/430; 524/88; 524/496; 524/388; 424/70.6

(57) ABSTRACT

The invention relates to an aqueous dispersion comprising at least one pigment and polyvinylpyrrolidone with a weight average molecular weight of less than 40,000 g/mol, its preparation method and its cosmetic uses.

### CROSS REFERENCE TO RELATED APPLICATIONS

The present application claims priority under 35 U.S.C. §119 to French Patent Application No. 10 58843, filed Oct. 27, 2010, which is incorporated herein by reference.

The present invention relates to a pigmentary aqueous dispersion and to its cosmetic uses.

Many cosmetic compositions comprise solids dispersed in the divided state, typically pigments. The more the pigments are dispersed homogeneously in the cosmetic composition and more they are present in a high proportion, more the color of the cosmetic composition is homogeneous and intense.

One of the objects of the present invention is to provide a pigmentary composition which may be used in cosmetics, which comprises a high pigment proportion, generally greater than or equal to 21% by weight, and which is stable over time.

The pigmentary composition of the invention is a pigmentary aqueous dispersion. In the sense of the present application, an aqueous dispersion (or aqueous suspension) is a continuous aqueous medium in which solid particles are dispersed. The aqueous medium is typically an aqueous solution comprising at least 40% preferably at least 50% of water, for example a buffer solution (such as a physiological buffer solution formed by the pair H₂CO₃/HCO₃⁻), preferably water.

In the present discussion, an aqueous dispersion is considered as stable over time if it may be kept generally for at least two months, preferably at least six months, at a temperature comprised between 5 and 45° C. while remaining homogeneous, i.e. the particles do not aggregate with each other and do not settle (no macroscopically observable sedimentation or phase shifting of the dispersion).

Thus, according to a first aspect, an object of the invention is an aqueous dispersion comprising at least one pigment and polyvinylpyrrolidone with a weight average molecular weight of less than 40,000 g/mol.

The inventors have now discovered that the use of polyvinylpyrrolidone (PVP) with a weight average molecular weight of less than 40,000 g/mol gives the possibility of obtaining a pigmentary aqueous dispersion comprising large amounts of pigment while being stable.

Without intending to be bound to a particular theory, it seems that it is possible to put forward that by using PVP with a weight average molecular weight of less than 40,000 g/mol it is possible to obtain a less viscous dispersion than when a PVP of higher molecular weight (for example PVP K30, with a weight average molecular weight of 60,000 g/mol) is used, which allows an increase in the concentration of pigment in the aqueous dispersion according to the invention. Thus, generally, the aqueous dispersion comprises at least 21% by weight (typically from 21 to 35%), notably at least 24% by weight (typically from 24 to 30%) of pigment based on the total amount of the aqueous dispersion.

Preferably, the PVP has a weight average molecular weight of less than or equal to 20,000 g/mol, notably less than or equal to 10 000 g/mol, and is typically of the order of 8,000 g/mol, such as PVP K15, advantageously commercially available.

Generally, the aqueous dispersion comprises from 1 to 20% by weight, notably from 3 to 10% of polyvinylpyrrolidone with a weight average molecular weight of less than 40,000 g/mol based on the total amount of the aqueous dispersion. These proportions are particularly suitable for obtaining a pigmentary aqueous dispersion comprising large amounts of pigment while being stable.

Typically, in the aqueous dispersion according to the invention, the ratio between the weight of polyvinylpyrrolidone with a weight average molecular weight of less than 40,000 g/mol and the weight of pigment is from 5 to 40%. These proportions are particularly suitable for obtaining a pigmentary aqueous dispersion comprising large amounts of pigment while being stable.

The pigment of the dispersion may be an organic or inorganic pigment.

The inorganic pigment may for example be selected from optionally surface-treated titanium dioxide, zinc dioxide, zirconium or cerium oxides, as well as iron or chromium oxides, manganese violet, ultramarine blue, chromium hydrate and ferric blue, and mixtures thereof.

The organic pigment may for example be selected from carbon black, such as D&C black no. 2, red organic pigments such as D&C Red no. 6, no. 7, no. 21, no. 27, no. 28, no. 30, no. 33, no. 36, no. 40, and cochineal carmine, blue organic pigments such as copper phthalocyanine and FD&C Blue no. 1 lacquer, and yellow organic pigments such as D&C Yellow no. 10, FD&C Yellow nos. 5 and 6 lacquers and mixtures thereof.

Carbon black is a pigment particularly suitable for applying the aqueous dispersion according to the invention. Carbon black may notably be high structure carbon black and/or carbon black having a specific surface area of more than 200 m²/g.

The main fundamental parameters defining the different marketed grades of carbon black are the size of the primary particles and of those of which are aggregated or agglomerated (fineness of the powder, structure) and its specific surface area.

The carbon black structure is determined by absorption of dibutyl phthalate (DBP). The amount of DBP absorbed by 100 g of carbon black is determined according to the ASTM D-2414 method. Thus, a carbon black with a low DBP index has a low structure consisting of a small number of primary particles which have merged in a compact way. Typically, the marketed blacks have a DBP absorption index comprised between 45 and 400 mL for 100 g of carbon black. A high DBP index, typically greater than 90 mL/100 g is synonym of carbon black of high structure, consisting of many elementary particles with abundant ramifications, branches, chains or strings.

The specific surface area of a carbon black may be determined by nitrogen adsorption (BET method). The specific surface area of a carbon black is generally comprised between 10 m²/g⁻¹ for coarse size particles and 500 m²·g⁻¹ for fine particles. The carbon black used as a pigment in the aqueous dispersion preferably has a specific surface area of more than 200 m²/g.

In addition to the PVP and to the pigment, the aqueous dispersion may comprise a compound comprising at least two hydroxyl functions and preferably free from ethylene oxide groups.

With this compound, it is possible to improve the stability of the aqueous dispersion.

The compound is preferably free from ethylene oxide groups of formula —CH_{2—}CH₂—O, in particular poly(ethylene oxide) groups. Indeed, during the synthesis of ethylene oxide, 1,4-dioxane is formed, which has a suspected carcinogenic effect. Thus, the compounds comprising ethylene oxide groups may comprise 1,4-dioxane, at least as traces, and it is sought to limit or even avoid their use in cosmetic compositions.

The compound comprising two hydroxyl functions may notably be a polyol, such as 1,2,3-propanetriol (glycerin), 1,2-propanediol, pinacol (2,3-dimethyl-2,3-butanediol), 1,2,3-butanetriol, 2,3-butanediol and sorbitol. Glycerin is particularly preferred because of its hydrating and antimicrobial nature.

The aqueous dispersion generally comprises from 5 to 45% by weight, typically from 20 to 40% by weight of compound comprising at least two hydroxyl functions based on the total amount of the aqueous dispersion, which generally corresponds to that the continuous aqueous medium of the aqueous dispersion is a 40/60 to 60/40, preferably 50/50 mixture of an aqueous solution (notably water) and of a compound comprising at least two hydroxyl functions (notably glycerin). These proportions are particularly suitable for obtaining a pigmentary aqueous dispersion comprising large amounts of pigment while being stable.

The aqueous dispersion may also comprise preservatives for example 2-phenoxyethanol or dehydroacetic acid. On the other hand, the use of paraben and/or of its derivative as a preservative is generally avoided because of their capability of activating receptors of estrogens, inducing possible action on fertility and estrogeno-dependent tumors.

In an embodiment, the aqueous dispersion consists in:

an aqueous medium, generally an aqueous solution typically water,

at least one pigment, notably carbon black, preferably high structure carbon black and/or carbon black having a specific surface area of more than 200 m²/g,

polyvinylpyrrolidone with a weight average molecular weight of less than 40,000 g/mol,

glycerin,

optionally preservatives such as 2-phenoxyethanol or dehydroacetic acid.

The aqueous dispersion may also comprise one or more cosmetic additives currently used in the art such as for example moisturizers, emollients, plasticizers, silicones, mineral fillers, clays, perfumes, peptizers, preservatives, proteins and vitamins, oils and/or natural extracts. Generally the aqueous dispersion is free of ionic surfactants.

The particles of the aqueous dispersion generally have a median size (D50) measured with a laser particle size analyzer, of more than 200 nm, typically greater than or equal to 400 nm. Typically, at least 90%, notably 95%, preferably 98%, or even the whole of the particles have a size measured with a laser particle size analyzer, of more than 100 nm. The aqueous dispersion is therefore mainly free of nanoparticles, which is an advantage since recent studies examine the question of the toxicity of the nanoparticles, in particular when they are used in cosmetic compositions, notably applied on the skin. Thus, the aqueous dispersion according to the invention is generally not a colloidal composition (which typically comprises particles for which the dimensions range from 2 to 100 nm).

Further, the particles of the aqueous dispersion generally have a median size (D50) measured with a laser particle size analyzer, of less than 500 μm, or even less than 100 μm. Indeed, sedimentation phenomena are limited or even avoided for such particles, and the cosmetic composition comprising the aqueous dispersion is more pleasant to the touch.

In a preferred embodiment, the aqueous dispersion comprises:

from 23 to 25 wt % of carbon black,

from 4 to 7 wt % of PVP K15,

from 30 to 40 wt % of water,

from 30 to 40 wt % of glycerin,

In a particularly preferred embodiment, the aqueous dispersion consists in:

from 23 to 25 wt % of carbon black,

from 4 to 7 wt % of PVP K15,

from 30 to 40 wt % of water,

from 30 to 40 wt % of glycerin,

from 0.1 to 15 wt % of phenoxyethanol, and

from 0.1 to 1.5 wt % of dehydroacetic acid,

the sum of the percentages being equal to 100.

A cosmetic composition comprising such aqueous dispersions actually gives the possibility of obtaining intense coloration when it is applied, notably on keratinous fibers.

According to a second aspect, an object of the invention is a method for preparing the aqueous dispersion as defined above, comprising a step for milling in a ball mill a mixture of at least one pigment and of polyvinylpyrrolidone with a weight average molecular weight of less than 40,000 g/mol in the presence of water.

The other optional components of the aqueous dispersion, as described above, may also be present during the milling step. The addition order of the components does not affect the nature of the obtained dispersion, and in particular has no influence on the size of the particles of the obtained dispersion.

Preferably, the balls of the ball mill have a diameter of more than 100 μm, generally comprised between 1 and 10 mm, typically of the order of 4 mm, which contributes to obtaining particles in the aqueous dispersion of median size (D50) greater than 100 nm, or even 1 μm, with the advantages mentioned above.

The milling time also has an influence on the size of the particles. Typically, when this time is too long, particles of nanometric size are obtained, which one seeks to avoid. The maximum milling time depends on the nature of the pigment. One skilled in the art knows how to adapt the milling time in order to have the desired particle size according to the nature of the pigment.

The method is generally free from a freeze-drying step.

The invention also relates to the aqueous dispersion obtainable with this method.

According to a third aspect, an object of the invention is the use of an aqueous dispersion as defined above or of an aqueous dispersion obtainable with the method described above for introduction into a cosmetic composition (or for preparing a cosmetic composition). The cosmetic composition may for example be a shampoo, a hair conditioner, a conditioner (hair conditioner balm), an eyeliner, a mascara, a foundation, an eye shadow, a blush or lipstick, preferably a shampoo, a hair conditioner, a conditioner or a mascara.

A cosmetic composition generally comprises an aqueous phase and a lipidic phase. The invention also concerns a process for the preparation of a cosmetic composition comprising a step of introducing an aqueous dispersion as defined above or of an aqueous dispersion obtainable with the method described above into the aqueous phase of a cosmetic composition followed by a step of mixing the obtained aqueous phase (i.e. the mixture of the aqueous phase of the cosmetic composition with the aqueous dispersion defined above) with the lipidic phase of the cosmetic composition.

The composition is particularly suitable for coloring keratinous fibers, such as hair and eyelashes.

Without intending to be bound to a particular theory, it seems that the specific size of the particles as defined above and the presence of polyvinylpyrrolidone with a weight average molecular weight of less than 40,000 g/mol promote deposition and adhesion of the pigment on keratinous fibers, leading to very good coloration. In particular, the inventors observe that cosmetic compositions free from PVP and/or having smaller particle sizes (nanometric sizes) applied on keratinous fibers lead to less significant coloration of the keratinous fibers.

The cosmetic composition may appear in any form (ointment, lotion, cream, gel, spray).

According to a fourth aspect, an object of the invention is a composition, preferably a cosmetic composition, comprising an aqueous dispersion as defined above.

According to a fifth aspect, an object of the invention is the use of polyvinylpyrrolidone with a weight average molecular weight of less than 40,000 g/mol as an additive in a cosmetic composition comprising an aqueous dispersion comprising a pigment, for increasing the pigment proportion of said composition.

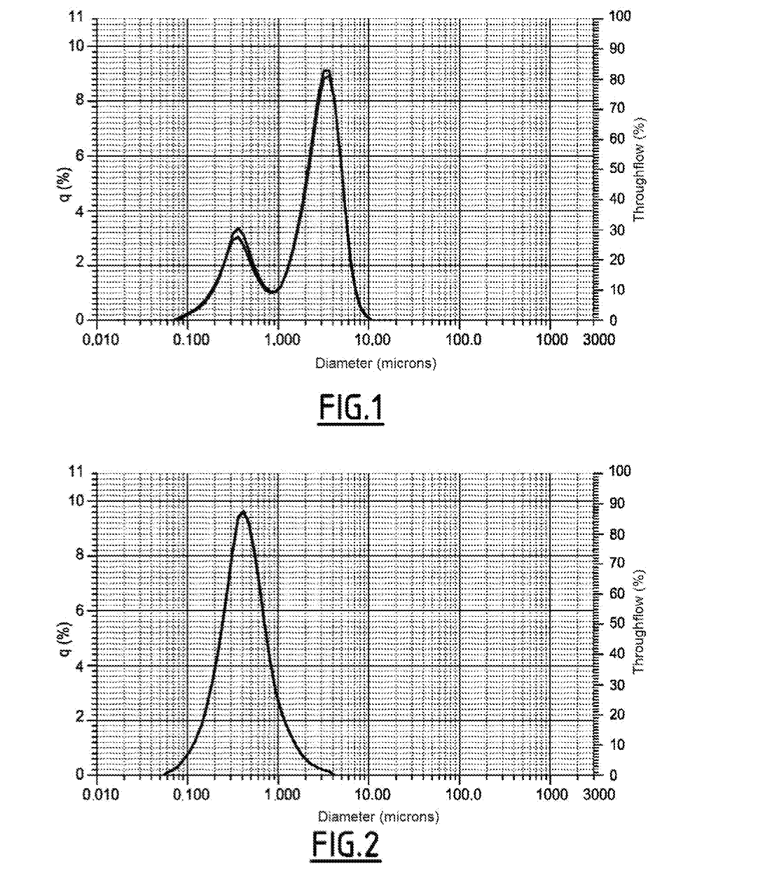

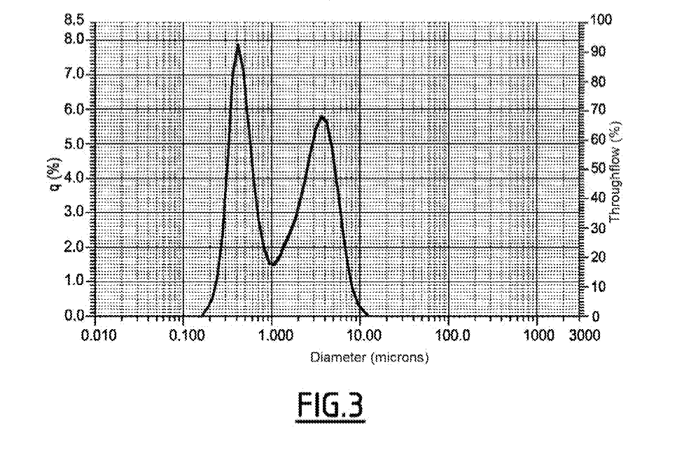

The invention is further illustrated with the examples hereafter, given with reference to FIGS. 1, 2 and 3 appended herein, which illustrate the particle size profile (percentage of particles versus the diameter of the particles in μm), of Examples A-1, A-2 and A-3 respectively.

### EXAMPLE

### Example A

### Aqueous Dispersions

### * Method for Preparing Aqueous Dispersions

Glycerin (Interchimie®) was introduced into a vertical ball mill of the COMEC® brand, the balls having a diameter of 4 mm and the maximum milling rate being 1800 revolutions per minute. 15% of the pigment Unipure Black LC902 (Sensient Cosmetic Technologies®) (a high structure carbon black having a DBP index of 100 mL/100 g) were then added. After homogenization in the mill, the water, the PVP and the preservatives (phenoxyethanol (Phenaxetol-Clariant®) and/or dehydroacetic acid (Geoguard 111A-Lonza®)) were added to the mixture. The remaining 85% of pigment were then added. For finishing, the mixture was milled at maximum speed for 15 minutes.

Five aqueous dispersions were prepared following this method.

Examples 1, 2 and 3 are examples of an aqueous dispersion according to the invention, comprising PVP K15 (with a weight average molecular weight of 8,000 g/mol) (ISP®).

Examples 4 and 5 are comparative examples in which a PVP of higher molecular weight (PVP K30 with a weight average molecular weight of 60,000 g/mol) (ISP®) was used.

* Characterization of the viscosity and of the particle size in order to evaluate the stability of the aqueous dispersions.

The viscosity is measured with a viscosimeter of the Brookfield DV-II+ P® brand and the suitable mobile.

The particle size is measured by laser diffraction according to Mie's theory with an apparatus of the HORIBA PARTICA LA-950 V2® type. The appended FIGS. 1, 2 and 3 illustrate the particle size (percentage of particles versus diameter of the particles in of Examples 1, 2 and 3 respectively.

The stability is considered to be good if the size of the particles of the dispersion and its viscosity remain constant over time.

TABLE 1
Compositions of the prepared aqueous dispersion and viscosity and particle size characteristics.

| | | | | Comparative | |
|---|---|---|---|---|---|
| | Examples | | | examples | |
| | 1 | 2 | 3 | 4 | 5 |
| Carbon black (%) | 22 | 30 | 24 | 22 | 30 |
| PVP K15 (%) | 5 | 6.81 | 5.45 | 0 | 0 |
| PVP K30 (%) | 0 | 0 | 0 | 5 | 6.81 |
| Water | 36.25 | 31.35 | 34.68 | 36.25 | 31.35 |
| Glycerin (%) | 36.25 | 31.35 | 34.68 | 36.25 | 31.35 |
| Phenoxyethanol | 0.5 | 0.5 | 0.7 | 0.5 | 0.5 |
| (%) | | | | | |
| Dehydroacetic | 0 | 0 | 0.5 | 0 | 0 |
| acid (%) | | | | | |
| Viscosity (cP) | 40 000 | 450 000 | 100 000 | 838 000 | Cakedⁱ⁾ |
| Stability | Good | Good | Good | Good | Poorⁱⁱ⁾ |
| Particle size: d50 | 2.5 | 0.42 | 1.37 | 4.32 | 5.15 |
| (μm) | | | | | |
| Particle size: | <0.3% | 1.30 | 0.00 | 0.00 | 0.00 |
| % of particles <100 nm | | | | | |

ⁱ⁾the dispersion cakes in order to attain a non-measurable viscosity (>50,000,000 cP).
ⁱⁱ⁾the size of the particles after 15 days at room temperature (25° C.) changes from 5.15 μm (t = 0) to 12.23 μm with occurrencies of aggregates with a size greater than 100 μm.

The comparison of the aqueous dispersions 1, 2 and 3 according to the invention and of the aqueous dispersions 4 and 5 comprising a PVP with a weight average molecular weight of more than 40,000 g/mol shows that the aqueous dispersions according to the invention are less viscous. It is possible to prepare a stable aqueous dispersion comprising 30% carbon black when PVP with a weight average molecular weight of 40,000 g/mol is used, which is not the case when a PVP with a weight average molecular weight of more than 40,000 g/mol is used (compare Examples 2 and 5).

Further, in the three compositions according to the invention, more than 98% of the particles have a size of more than 100 nm.

### Example B

### Cosmetic Composition Comprising an Aqueous Dispersion

The Examples hereafter relate to cosmetic compositions comprising the aqueous dispersion according to the invention.

### B.1. Hair Conditioner Care Products with “Black Reflections”

Two hair care products were made by using the dispersion of Example 3 of Example A and according to the formulation and operating procedure described below:

TABLE 2
composition of the two hair care products

| | | | | % (w/w) | |
|---|---|---|---|---|---|
| Phase | Trade name | Supplier | Cosmetic name (INCI) | Ex 1 | Ex 2 |
| A | Oxocera | Sensient | Cetyl alcohol - ceteareth-25 | 7.0 | 7.0 |
| | | Cosmetic | | | |
| | | Technologies | | | |
| | Covasterol | Sensient | Glyceryl isostearate - isostearyl | 4.0 | 4.0 |
| | | Cosmetic | alcohol - Brassica campestris | | |
| | | Technologies | (rapeseeds) sterols - | | |
| | | | Butyrospermum parkii butter - | | |
| | | | candelilla cera | | |
| | Natpure Feel-M | Sensient | Isopropyl palmitate - isostearyl | 2.0 | 2.0 |
| | Eco | Cosmetic | isostearate - octyldodecanol - | | |
| | | Technologies | octyldodecyl myristate | | |
| B | Pure water | | Aqua | 58 | 58 |
| | Dehyquart A-CA | Cognis | Cetrimonium chloride | 4.5 | 4.5 |
| C | Pure water | | Aqua | 22.75 | 22.55 |
| | Natpure Trap TS | Sensient | Sodium phytate - sodium | 0.5 | 0.5 |
| | | Cosmetic | citrate | | |
| | | Technologies | | | |
| | Phenoxyethanol | | Phenoxyethanol | 0.5 | 0.5 |
| | Carbon black | Sensient | CI 77266 - Glycerin - Aqua - | 0.75 | 0.75 |
| | dispersion | Cosmetic | PVP - phenoxyethanol - | | |
| | (Example A-3) | Technologies | dehydroacetic acid | | |
| | 306020 Arianor | Sensient | Basic Blue 99 - Basic Brown | — | 0.2 |
| | Ebony | Cosmetic | 16 - Acid Violet 43 - Basic Red | | |
| | | Technologies | 76 - Basic Yellow 57 | | |

The operating procedure which was followed is the following:

1. Heating of phase A to 70-80° C.

### 2. Heating of B to 70-80° C.

3. Pour the phase B into the phase A and stir so as to obtain a homogeneous emulsion.
4. Add the phase C and mix till room temperature is reached.

When one of these two conditioner care products is applied on bleached hair and then rinsed, the black pigment is deposited on the hair. The hair is therefore intensely colored in black after application.

### B.2. Shampoo with “Black Reflections”

Two shampoos were made by using the dispersion of Example 3 of Example A and following the formulation and operating procedure described below:

TABLE 3
composition of the two shampoos

| | | | | % (w/w) | |
|---|---|---|---|---|---|
| Phase | Trade Name | Supplier | Cosmetic name (INCI) | Ex 1 | Ex 2 |
| A | Pure water | | Aqua | 16.6 | 16.6 |
| | Natpure Trap TS | Sensient | Sodium phytate - sodium citrate | 0.5 | 0.5 |
| | | Cosmetic | | | |
| | | Technologies | | | |
| | Carbopol Aqua SF-1 | Noveon | carbomer | 10.0 | 10.0 |
| | Plantacare 1200 UP | Cognis | Lauryl glucoside | 8.0 | 8.0 |
| | Plantapon LCG | Cognis | Sodium lauryl glucose | 17.0 | 17.0 |
| | Sorb | | carboxylate - lauryl glucoside | | |
| | Dehyton K Cos | Cognis | Cocamidopropyl betaine | 16.0 | 16.0 |
| B | Pure water | | Aqua | 15.0 | 15.0 |
| | Jaguar C13S | Rhodia | Guar hydroxypropyltrimonium | 0.1 | 0.1 |
| | | | chloride | | |
| | Triethanolamine | | Ethanolamine | 2.2 | 2.2 |
| | 99% | | | | |
| C | Natpure Feel-M Eco | Sensient | Isopropyl palmitate - isostearyl | 1.0 | 1.0 |
| | | Cosmetic | isostearate - octyldodecanol - | | |
| | | Technologies | octyldodecyl myristate | | |
| | Phenoxyethanol | | phenoxyethanol | 0.5 | 0.5 |
| D | Carbon black | Sensient | CI 77266 - Glycerin - Aqua - | 1.5 | 1.5 |
| | dispersion | Cosmetic | PVP - phenoxyethanol - | | |
| | (Example A-3) | Technologies | dehydroacetic acid | | |
| | Water | | | 11.6 | 11.5 |
| | 306020 Arianor | Sensient | Basic Blue 99 - Basic Brown 16 - | — | 0.1 |
| | Ebony | Cosmetic | Acid Violet 43 - Basic Red 76 - | | |
| | | Technologies | Basic Yellow 57 | | |

The operating procedure which was followed is the following:

1. Mix the different ingredients of phase A until a homogeneous mixture is obtained.
2. Prepare phase B and pour into phase A.
3. Add phase C, mix.
4. Add phase D, homogenize,

When one of these two shampoos is applied on hair and then rinsed, the black pigment is deposited on the hair. The hair is therefore intensely colored in black after the application.

### B.3. Eyeliner

Two eyeliners were made by using the dispersion of Example 3 of Example A and by following the formulation and the operating procedure described below:

TABLE 4
composition of the two eyeliners

| | | | | % (w/w) | |
|---|---|---|---|---|---|
| Phase | Trade name | Supplier | Cosmetic name (INCI) | Ex 1 | Ex 2 |
| A | Monopropylene | | Propylene glycol | 1.40 | 1.40 |
| | glycol | | | | |
| | Thickagent LC | Sensient | Xanthan gum - hectorite - | 0.70 | 0.70 |
| | | Cosmetic | cellulose | | |
| | | Technologies | | | |
| | Pure water | | Aqua | 52.60 | 52.60 |
| | Phenoxyethanol | | Phenoxyethanol | 0.30 | 0.30 |
| B | Carbon black | Sensient | CI 77266 - Glycerin - Aqua - | 30.00 | 30.00 |
| | dispersion | Cosmetic | PVP - phenoxyethanol - | | |
| | (Example A-3) | Technologies | dehydroacetic acid | | |
| C | Covacryl MS11 | Sensient | Acrylates copolymer | 15.00 | |
| | | Cosmetic | | | |
| | | Technologies | | | |
| | Covacryl P12 | Sensient | Acrylates copolymer | | 15.00 |
| | | Cosmetic | | | |
| | | Technologies | | | |

The operating procedure that was followed is the following:

1. Mix the Thickagent LC in the monopropylene glycol.
2. Pour this mixture into the water containing the preservative, with stirring, an as to obtain a homogeneous gel.
3. Add phase B with stirring. Properly mix.
4. Add phase C. Homogenize.

When one of these two eyeliners is applied on eyelashes the black pigment is deposited on the eyelashes, leading to intense black coloration of the eyelashes.

1. An aqueous dispersion comprising at least one pigment and polyvinylpyrrolidone with a weight average molecular weight of less than 40,000 g/mol.

2. The aqueous dispersion according to claim 1, wherein the polyvinylpyrrolidone has a weight average molecular weight of less than or equal to 10,000 g/mol.

3. The aqueous dispersion according to claim 1, wherein the ratio between the weight of polyvinylpyrrolidone with a weight average molecular weight of less than 40,000 g/mol and the pigment weight is from 5 to 40%.

4. The aqueous dispersion according to claim 1, wherein at least 90% of the particles have a size measured with a laser particle size analyzer, of more than 100 nm.

5. The aqueous dispersion according to claim 4, wherein at least 98% of the particles have a size measured with a laser particle size analyzer, of more than 100 nm.

6. The aqueous dispersion according to claim 1, wherein the pigment is an inorganic pigment selected from titanium dioxide, zinc dioxide, zirconium or cerium oxides, iron or chromium oxides, manganese violet, ultramarine blue, chromium hydrate and ferric blue and mixtures thereof.

7. The aqueous dispersion according to claim 1, wherein the pigment is an organic pigment selected from carbon black, such as D&C Black no. 2, red organic pigments such as D&C Red no. 6, no. 7. no. 21, no. 27, no. 28, no. 30, no. 33, no. 36, no. 40 and cochineal carmine, blue organic pigments, such as copper phthalocyanine and FD&C Blue no. 1 lacquer, and yellow organic pigments such as D&C Yellow no. 10, FD&C Yellow nos. 5 and 6 lacquers and mixtures thereof.

8. The aqueous dispersion according to claim 7, wherein the pigment is high structure carbon black and/or carbon black having a specific surface area of more than 200 m²/g.

9. The aqueous dispersion according to claim 1, further comprising a compound comprising at least two hydroxyl functions, preferably free from ethylene oxide groups.

10. The aqueous dispersion according to claim 9, wherein the compound is a polyol, preferably glycerin.

11. The aqueous dispersion according to claim 1, comprising at least 21% by weight, notably at least 24% by weight, of pigment based on the total amount of the aqueous dispersion.

12. A method for preparing an aqueous dispersion according to claim 1, comprising a step for milling in a ball mill a mixture of at least one pigment and polyvinylpyrrolidone with a weight average molecular weight of less than 40,000 g/mol in the presence of water.

13. A cosmetic composition comprising an aqueous dispersion according to claim 1.

14. A cosmetic composition comprising an aqueous dispersion obtainable with the method according to claim 12.
